(19) United States
(12) Plant Patent Application Publication
(10) Pub. No.: US 2002/0066126 P1
(43) Pub. Date: May 30, 2002

(54) MINIATURE ROSE PLANT NAMED 'SAVAGALA'

(76) Inventor: John Saville, Newburyport, MA (US)

Correspondence Address: John M. Saville, Nor ' East Miniature Roses, Inc., P.O. Box 307, Rowley, MA, 01969-0607, US

(21) Appl. No.: 09/727,064

(22) Filed: Nov. 30, 2000

(51) Int. Cl. A01H 5/00

(52) U.S. Cl. PLT/121

(57) ABSTRACT

SAVagala is a new and distinct variety of miniature rose plant primarily identified by it's magenta pink, double blooms of exhibition form, borne singly and in small sprays on a low growing, upright yet spreading, well branched plant with semi-glossy dark green foliage. It is a recurrent bloomer throughout the growing season and can be grown quite satisfactorily in a greenhouse, outdoors in containers or as a hardy garden perennial. Blooms are long lasting on the plant and as cut flowers.

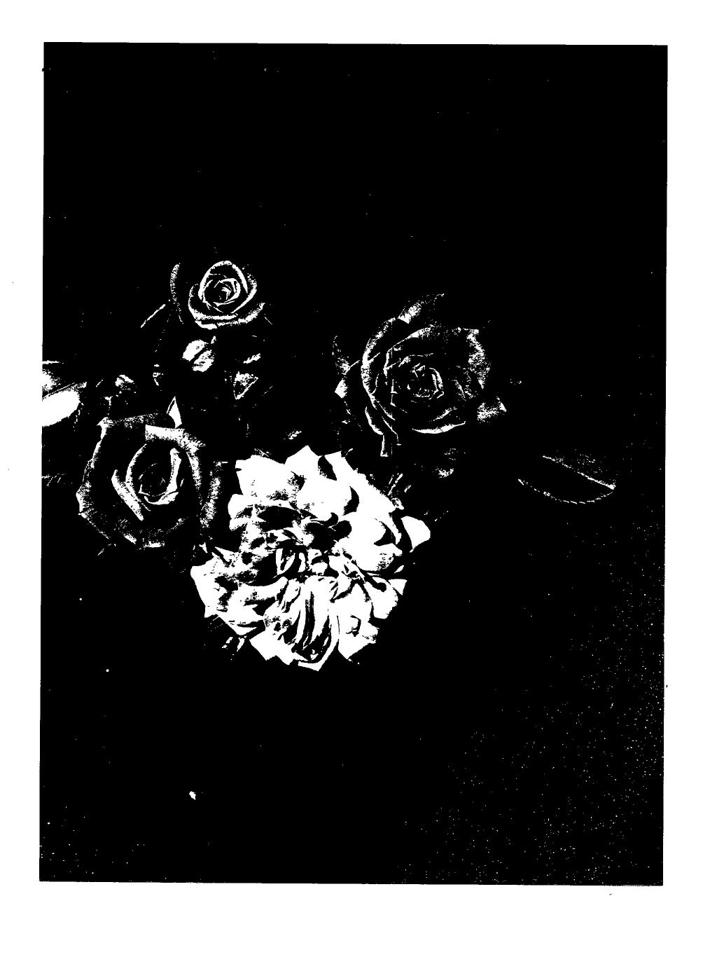

### CROSS REFERENCE

This present invention relates to a new and distinct variety of hardy, bush type plant of the miniature rose class, whose bud form and flower color are similar to those of SAVawin (U.S. Plant Pat. No. 5,691), a miniature rose of spinel red coloring. The flowers of this new invention have more petals and are a slightly more pink and darker color than SAVawin.

### BACKGROUND OF THE INVENTION

This present invention is a new and distinct variety of miniature rose plant which was created in a greenhouse in Rowley, Mass., by crossing the following two rose plants:

The seed parent is SAVajinks (U.S. Plant Pat. No. 8,412), a miniature rose of magenta red and white flowers and exhibition form.

The pollen parent is an unnamed and unintroduced seedling.

This invention is a rose plant of the miniature rose class (Rosa chinensis ‘minima’) having flowers and foliage typical of specimens in that class, and which can be grown quite satisfactorily in a greenhouse, outdoors in containers and as a hardy garden perennial, with protection recommended through the winter in severe climates.

The varietal denomination of this new rose is SAVagala

Asexual reproduction by cuttings of this new variety shows that all distinguishing characteristics of this rose continually come true to form.

### SUMMARY OF THE INVENTION

The present invention relates to a new and distinct variety of hardy dwarf bush type rose plant of the miniature class; the variety being primarily characterized—as to novelty—by it's vibrant magenta pink blooms of good exhibition (hybrid tea) form on a low growing plant.

The variety is further characterized by:

An abundance of these bright flowers borne most often in loose sprays of 3 to 7 flowers but also borne singly.

A plant which has good vigor, is well branched, growing 16-20 inches tall and spreading 20 inches or more with abundant dark green semi-glossy foliage.

No fragrance.

A plant which grows and blooms satisfactorily both in the greenhouse and outdoors: provides decoration as a garden perennial, specimen plant or in containers and is good as a cut flower.

### BRIEF DESCRIPTION OF THE DRAWING

The accompanying color photograph show specimens of the flower of this new variety in different stages of development as grown and observed in September, under plastic in Essex County, Mass.

### BOTANICAL DESCRIPTION OF THE PLANT

Following is a detailed description in outline form pertaining specifically to this new and distinct variety of miniature rose plant. All major color plate identifications made are referring to The Royal Horticulture Society Chart except where common terms of color definition are employed.

Class: Miniature rose

Flowers:

Blooming habit.—Recurrent.

Borne.—Most often in sprays of 3 to 7 and also borne singly.

Bud:

Size.—Medium.

Form.—Ovate.

Color.—When sepals first divide — usually a dark to medium magenta pink, near 61 C.

Sepals.—color — a medium yellow green, near 144A. characteristics — very finely bearded and lanceolate, extending just beyond the tip of the bud, curling back as the flowers open and remaining curled back and attached until hips start to ripen.

Peduncle.—Length — averaging 1½ inch.

Aspect.—Straight.

Strength.—Erect.

Color.—A light yellow green, near 144C and flushed lightly with near 63C.

Bloom:

Size.—When fully expanded — 1½ to 2 inches.

Form.—High centered at first, becoming flat; petals remain loosely rolled outward.

Petalage.—20 to 25.

Petaloids.—Usually many.

Fragrance.—None.

Petals:

Texture.—Medium.

Appearance.—Inside — velvety, outside — satiny.

Form.—Broad spatulate.

Arrangement.—Arranged shingle-like.

Color.—During the first few days — a dark to medium magenta pink, near 61 C and the reverse being a bit more subdued, near 63A. As the bloom ages it becomes lighter, near 68A, and flushed with a deeper shading of near 64C, and near 66D at the center. Petals become white at the point of attachment.

General Tonality.—Magenta pink.

Reproductive organs:

Stamens: filaments and anthers:

Arrangement.—Regularly arranged around styles.

Quantity.—Few.

Filaments.—Length — very short.

Anthers.—Color — a medium yellow.

Pollen:

Color.—Medium yellow.

Quantity.—Little.

Pistils, styles and stigmas:

Quantity.—Average.

Styles.—Thin, short, uneven length, color — medium red.

Stigmas.—Near white.

Hips:

Shape.—Globular.

Size.—Up to ¾ inch.

Color.—Differing shades of orange, depending on how intensely the sun hits an area — near 169A, 169B and 26A

Plant:

Habit.—Upright and spreading.

Growth.—Vigorous and well branched, maturing at 16-20 inches in height and spreading to 20 inches or more.

Foliage:

Size.—Medium.

Quantity.—Abundant.

Color.—New foliage — dark green on the upper surface, between 137A and 147A and a medium green on the underside, near 147B.

Older foliage.—A dark green, near 147A, on the upper surface and a medium green, near 147B, on the reverse.

Texture.—Semi-glossy on the upper surface and matte on the underside.

Leaflets.—Usually 5 leaflets, but also may have 3 or 7.

Shape.—Oval, pointed.

Edge.—Fine, uneven serration.

Leaf stem.—The upper surface being a medium green, near 147B, and the underside of the leaf stem having 1 to 3 prickles and being a medium shade of yellow green, near 144B.

Stipules.—Finely bearded, averaging ½ inch in length.

Resistance.—Average resistance to black spot and downy mildew, no rust observed.

Wood.—Smooth with new wood being near 144B and flushed with a deep burgundy, near 183B; and old wood is a medium olive green, near 146B.

Thorns.—Quantity on main stalks — few, and on laterals — many.

Form.—A flat base tapering to a fine point, thin, straight and slightly angled downward, and being of medium length.

Color when young.—a light shade of magenta, between 185B and 185C

Winter Hardiness—Needs protection in cold climates.

The following is claimed:

A new and distinct variety of hardy miniature rose plant substantially as illustrated and described, characterized by it's vibrant, deep magenta pink blooms of exhibition form on a low growing plant with dark green, semi-glossy foliage.

1. I claim a new and distinct variety of hardy miniature rose plant substantially as illustrated and described, characterized by it's vibrant, deep magenta pink blooms of exhibition form on a low growing plant with dark green, semi-glossy foliage.
